(19) United States
(12) Patent Application Publication
(10) Pub. No.: US 2010/0159495 A1
(43) Pub. Date: Jun. 24, 2010

(54) PROSECC AN MEANS FOR ENZYMATIC DETERMINATION OF ACETATE

(76) Inventor: Rolf Vormbrock, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/161,046

(22) PCT Filed: Dec. 20, 2006
(86) PCT No.: PCT/EP2006/012299

§ 371 Date: Jul. 16, 2008

(30) Foreign Application Priority Data
Jan. 17, 2006 (DE) 10 2006 002 165.7

(51) Int. Cl. C12Q 1/32; C12Q 1/25

(52) U.S. Cl. 435/26; 435/4

(57) ABSTRACT

The invention relates to a method and means for the enzymatic determination of acetate. The method according to the invention facilitates for the first time reflectometric detection by means of a test strip.

The invention relates to a method and means for the enzymatic determination of acetate. The method according to the invention facilitates for the first time reflectometric detection by means of a test strip.

The determination of acetate plays an important role in many areas, such as, for example, in industry for monitoring of fermentation processes or for determination of the purity of reagents, in the foods industry for determination of the acetate content of foods and drinks, or in winemaking.

Many methods for the determination of acetate are known in the prior art. In some cases, great technical complexity is necessary for carrying out these methods, for example in wine analysis for the determination of volatile acids, which represent a quality feature and whose principal constituent is acetic acid. Enzymatic determination methods are therefore generally preferred since they can be carried out rapidly and simply. These methods are wet-chemical methods. A review of the most important methods for the determination of acetate is given in H. U. Bergmeyer (ed.), Methods of Enzymatic Analysis (3rd Edition, Weinheim 1984, Vol. 6, pages 628-645).

The commonest method at present for the determination of acetate is a wet-chemical, photometric method using acetyl-CoA synthetase (described in H.-O. Beutler [Acetate, Determination with Acetyl-CoA Synthase, in H. U. Bergmeyer (ed.), Methods of Enzymatic Analysis, 3rd Edition, Weinheim 1984, Vol. 6 p 639-645]). The photometric determination is based on the detection of NADH.

Since photochemical determination is both complex in terms of equipment and is usually highly dependent on the inherent colour of the sample, it would be desirable to be able to carry out a determination of acetate in which the detection takes place reflectometrically via a strongly coloured product.

The object of the present invention was therefore to provide a determination of acetate which is faster and simpler to carry out than the titrimetric determination of volatile acids or the known photometric method using acetyl-CoA synthase.

It has been found that the known photometric method using acetyl-CoA synthase cannot be coupled directly to a colour reaction which converts the NADH formed into a coloured formazane, for example with reduction of a tetrazolium salt. This is because it is not only the NADH formed by the acetate determination that is converted in such a reaction. Instead, irrespective of the acetate concentration, sufficient formazane is formed until one of the components tetrazolium salt or malate employed in the test has reacted completely. Quantitative determination of acetate is thus not possible in this way.

It has furthermore been found that reflectometric determination of acetate is possible if the acetate determination is carried out in two steps, where, in a first step, a reaction is carried out on a test strip by the method of H.-O. Beutler [Acetate, Determination with Acetyl-CoA Synthase in H. U. Bergmeyer (ed.), Methods of Enzymatic Analysis, 3rd Edition, Weinheim 1984, Vol. 6, pp. 639-645]. In a second step, the concentration of the NADH formed is subsequently determined on a test strip by means of phenazine methosulfate or a derivative thereof with reduction of a tetrazolium salt to a coloured formazane. In this procedure, the influence of the malate dehydrogenase from the first reaction step is reduced to such an extent that it does not result in falsification of the result. After the end of the reaction on the test strip, the formazane colour formed is measured reflectometrically as remission. The concentration of the acetate in the sample can be calculated therefrom via a calibration curve. The present invention therefore relates to a method for the determination of the acetate content of a sample, comprising at least the following method steps:
a) provision of a sample whose acetate content is to be determined
b) dilution and setting of the pH of the diluted sample solution by means of a buffer solution which comprises malic acid
c) bringing the dilute sample into contact with a test strip which comprises at least a tetrazolium salt, ATP, coenzyme A, NAD, malate dehydrogenase, citrate synthase and acetyl CoA synthetase;
d) incubation of the test strip from step c)
e) bringing the test strip from step d) into contact with a liquid reagent composition which comprises at least phenazine methosulfate or a derivative thereof.
f) incubation of the test strip from step e)
g) determination of the acetate content with reference to the colour development forming

In a particular embodiment, a buffer which uses triethanolamine as buffer substance is used in step b)

In a further preferred embodiment, a test strip which comprises nitro blue tetrazolium chloride as tetrazolium salt can be used in step c).

In another embodiment according to the invention, the sample provided in step a) is a beverage.

In a further preferred embodiment, the reagent composition from step e) has a pH between 2.5 and 5.5.

In a preferred embodiment, the determination of the acetate content in step g) is carried out reflectometrically.

The present invention also relates to a test kit for the determination of the acetate content of a sample, at least comprising
a) a buffer solution which comprises malic acid in addition to the actual buffer substance.
b) a test strip which comprises at least a tetrazolium salt, ATP, coenzyme A, NAD, malate dehydrogenase, citrate synthase and acetyl CoA synthetase;
c) a liquid reagent composition which comprises at least phenazine methosulfate or a derivative thereof.

In a preferred embodiment, buffer solution, test strips and reagent compositions comprise, independently of one another, buffers and stabilisers.

In a preferred embodiment, the test strip comprises nitro blue tetrazolium chloride as tetrazolium salt.

In another preferred embodiment, the reagent composition from step e) has a pH between 2.5 and 5.5.

In the description of the invention, the following abbreviations are used:

NAD nicotinamide adenine dinucleotide (oxidised form)

NADH nicotinamide adenine dinucleotide (reduced form)

NBT nitro blue tetrazolium chloride

In accordance with the invention, is a sample is typically liquid. It may be, for example, a solution or suspension, which generally comprises water or a buffer solution as the predominant solvent. Solid samples are therefore firstly dissolved, suspended or extracted in or with water or a buffer solution. Examples of samples are beverages, such as fruit or vegetable juices, alcoholic beverages, in particular wines, or blood. Depending on the nature of the sample and the acetate content thereof, it may be advantageous to dilute the sample with water before carrying out the determination according to the invention.

Analysis using solid, sorptive supports, so-called test sticks or test strips, has recently been increasing in importance. The main advantages of these dry-chemical methods include, in particular, simple handling and straight-forward disposal owing to the small amounts of reagent. All or the majority of the reagents necessary for the determination reaction are embedded in corresponding layers of a solid, sorptive or swellable support to which the sample is applied. After contact of the reaction zone with the sample, the determination reaction proceeds. The colour formed is a measure of the amount of analyte to be determined and can be evaluated visually, i.e. semi-quantitatively, or quantitatively using simple reflectometers.

Sorptive supports which can be used for the test strips of the test kit according to the invention are all materials which are usually in use for dry-chemical tests of this type. The most widespread is the use of filter paper, but other sorptive cellulose or plastic products can also be employed.

The sorptive supports are impregnated with impregnation solutions in a known manner. The impregnated and dried supports can be cut to suitable size and adhesively bonded or heat-sealed to support films or foils in a known manner for the production of test strips.

The sorptive support to which the determination reagents are applied usually does not cover the entire test strip, but instead merely a zone of the test strip. In this way, it is possible to combine not only one zone having one composition of determination reagents, but instead a plurality of zones having identical or different compositions on a single test strip. The region of the test strip to which the reagents necessary for the determination of an analyte or for the recording of a blank value are applied on a sorptive support is therefore referred to in accordance with the invention as a zone.

In accordance with the invention, acetate denotes acetic acid and salts of acetic acid.

In accordance with the invention, ATP denotes adenosine triphosphate and salts of this compound which can be converted by the enzymes employed in accordance with the invention in the same way, such as, for example, adenosine triphosphate disodium salt.

In accordance with the invention, coenzyme A denotes coenzyme A salts of this compound which can be converted by the enzymes employed in accordance with the invention in the same way, such as, for example, coenzyme A trilithium salt.

In accordance with the invention, phenazine methosulfate denotes 5-methylphenazinium methylsulfate or derivatives thereof, such as, for example, 1-methoxy-5-methylphenazinium methylsulfate.

The acetate content determination according to the invention is carried out in two steps. In a first step, acetate is converted on a test strip in accordance with the method of H.-O. Beutler [Acetate, Determination with Acetyl-CoA Synthase in H. U. Bergmeyer (ed.), Methods of Enzymatic Analysis, 3rd Edition, Weinheim 1984, Vol. 6, pp. 639-645]. The NADH forming in the process is then consumed in a second step by converting a tetrazolium salt located on the test strip into a coloured formazane by addition of phenazine methosulfate or derivatives of this compound, where this reaction is preferably carried out at pH values below pH 6.0.

The buffer solution employed for the sample dilution comprises at least a buffer substance and malic acid. Preferred concentration ranges of these components in the solutions according to the invention are in the range from:

| | |
|---|---|
| triethanolamine | 0.2 to 1.6 mol/l |
| and | |
| L-malic acid | 20 to 100 mmol/l. |

Particularly preferred concentrations are:

1.0 to 1.5 mol/l of triethanolamine
50 to 80 mmol/l of L-malic acid

The test strip employed in the first reaction step comprises at least a tetrazolium salt, ATP, coenzyme A, NAD, malate dehydrogenase, citrate synthase and acetyl CoA synthetase.

To this end, a test strip is impregnated by known methods with one or more impregnation solutions which comprise the corresponding substances.

The concentrations of the individual constituents in the impregnation solutions correspond to the concentrations which can also be used for wet-chemical acetate determination in accordance with the prior art.

Preferred concentration ranges are

1.0 to 5.0 mmol/l of magnesium chloride or sulfate
10 to 25 mmol/l of adenosine triphosphate disodium salt
10 to 30 mmol/l of nicotinamide adenine dinucleotide
0.8 to 1.5 mmol/l of coenzyme A trilithium salt
20 to 100 U/ml of malate dehydrogenase
5 to 25 U/ml of citrate synthase
1.0 to 5.0 U/ml of acetyl-CoA synthetase

Particularly preferred concentrations are:

2.0 mmol/l of magnesium chloride
15 mmol/l of adenosine triphosphate disodium salt
12 mmol/l of nicotinamide adenine dinucleotide
1.1 mmol/l of coenzyme A trilithium salt
43 U/ml of malate dehydrogenase
14 U/ml of citrate synthase
1.6 U/ml of acetyl-=CoA synthetase

The tetrazolium salt is typically present in the impregnation solutions in a concentration between 0.5 to 5.0 g/l, preferably between 500 mg and 2 g/l. Suitable tetrazolium salts are NBT or MTT (3-(4,5-dimethylthiazol-2-yl)-2,5-diphenyl-2-tetrazolium bromide).

In general, the impregnation solutions are aqueous solutions, which may comprise a proportion of one or more alcohols, such as methanol or preferably ethanol. The impregnation solutions furthermore typically comprise salts, buffers and/or stabilisers. The person skilled in the art in the area of test strip manufacture knows the substances which are suitable for this purpose.

For the test strip according to the invention, the impregnation solution preferably has a pH between 7.0 and 7.5, particularly preferably 7.2. This pH can be produced, for example, using a triethanolamine buffer or a BIS-TRIS-propane buffer.

The test strip can be brought into contact with the sample, for example, by brief dipping into the dilute sample or dropwise application of the dilute sample to the test strip.

In order to ensure that the acetate in the sample has reacted as completely as possible, the sample is incubated on the test strip. The incubation time is typically between 30 seconds and 20 minutes, preferably 5 to 15 minutes. The incubation is preferably carried out at temperatures between 18 and 35° C., particularly preferably at room temperature.

In accordance with the invention, the concentration of the NADH formed is subsequently determined in a second step (method steps e) to f) of the method according to the invention). This is carried out by bringing the test strip from method step d) into contact with a reagent combination which comprises at least phenazine methosulfate or a suitable derivative thereof.

This reagent composition is in liquid form, typically as a solution. Preferred concentration ranges of phenazine methosulfate or of a suitable derivative are between 25 and 400 μg/ml, particularly preferably between 60 and 150 μg/ml. In general, the reagent composition additionally comprises buffers and/or stabilisers. The preferred pH range of the reagent composition is between pH 2 and pH 6, particularly preferably between pH 3 and 5. Suitable buffers for setting this range are buffer substances having a pKₐ value in the range from 2.0 to 6.5, preferably salts of citric acid.

In order to ensure that the NADH has reacted as completely as possible, the test strip is incubated after being brought into contact with the reagent composition. The incubation time is typically between 30 seconds and 10 minutes, preferably about 5 minutes. The incubation is preferably carried out at temperatures between 18 and 35° C., particularly preferably at room temperature.

After the end of the reaction, the intensity of the colour of the formazane is determined. This can be carried out visually, for example semi-quantitatively by comparison with a colour chart, or quantitatively, for example by means of a reflectometer. The concentration of the acetate in the sample can be calculated therefrom via a calibration curve.

The present invention also relates to a test kit for carrying out the method according to the invention.

The test kit comprises at least
a) a buffer solution for dilution of the sample and setting of the pH, which comprises at least a buffer substance and malic acid;
b) a test strip which comprises at least a tetrazolium salt, ATP, coenzyme A, NAD, malate dehydrogenase, citrate synthase and acetyl CoA synthetase;
c) a liquid reagent composition which comprises at least a buffer substance for setting the pH and phenazine methosulfate or a derivative thereof.

The test kit furthermore preferably contains a description for carrying out the method according to the invention.

With respect to the composition of the reagent compositions and the preferred embodiments, reference is made to the information given in the description of the method according to the invention.

The determination method provided in accordance with the invention enables acetic acid concentrations from 40-400 mg/l to be determined in the form of acetate. Higher concentrations can be determined after corresponding dilution with water.

The method according to the invention and the test kit according to the invention thus offer a simple, rapid and sensitive method for the enzymatic determination of acetate. The simple method procedure with the aid of a test strip enables the determination to be carried out even by untrained personnel without complex equipment and without handling of toxic chemicals.

Even without further comments, it is assumed that a person skilled in the art will be able to utilise the above description in the broadest scope. The preferred embodiments and examples should therefore merely be regarded as descriptive disclosure which is absolutely not limiting in any way.

The complete disclosure content of all applications, patents and publications mentioned above and below is incorporated into this application by way of reference.

### EXAMPLE

Determination of Acetic Acid in Wine

Test strips having two paper zones for the determination of acetic acid are produced and impregnated in the first step with a solution of nitro blue tetrazolium chloride in triethanolamine buffer pH 7.2.

The solution has the following composition:

45 mmol/l of triethanolamine buffer pH 7.2
60 g/l of ethanol
1 g/l of nitro blue tetrazolium chloride fillers and stabilisers

A solution of the following composition is subsequently applied to the two zones:

2.0 mmol/l of magnesium chloride hexahydrate
15 mmol/l of adenosine triphosphate disodium salt
12 mmol/l of nicotinamide adenine dinucleotide
1.1 mmol/l of coenzyme A trilithium salt
43 U/ml of malate dehydrogenase
14 U/ml of citrate synthase
1.6 U/ml of acetyl-CoA synthetase fillers and stabilisers

After drying, these test strips are stored under dry conditions at 2° C. to 8° C.

The determination is carried out as follows:

1.0 ml of buffer solution (60 mmol/l of malic acid in 1.4 mol/l of triethanolamine buffer pH 8.0) is added to 1.0 ml of a pre-diluted sample, and the mixture is diluted with 8.0 ml of water. A test stick is dipped into this solution. Excess liquid is removed from the test strip, and the test strip is incubated at room temperature for 10 minutes.

One drop of a solution of 1-methoxy-5-methylphenazinium methylsulfate in a citrate buffer solution pH 3.5 is subsequently applied dropwise to each paper zone, and, after a further reaction time of 5 minutes, the colour formed in the paper zones is measured reflectometrically. The acetic acid concentration is calculated via a calibration curve.

1. Method for the determination of the acetate content of a sample, comprising at least the following method steps:
a) provision of a sample whose acetate content is to be determined
b) dilution and setting of the pH of the diluted sample solution by means of a buffer solution which, besides the buffer substance, comprises at least malic acid
c) bringing the sample into contact with a test strip which comprises at least a tetrazolium salt, ATP, coenzyme A, NAD, malate dehydrogenase, citrate synthase and acetyl CoA synthetase;
d) incubation of the test strip from step c)
e) bringing the test strip from step d) into contact with a liquid reagent composition which comprises at least phenazine methosulfate or a derivative thereof.
f) incubation of the test strip from step e)
g) determination of the acetate content with reference to the colour development forming.

2. Method according to claim 1, characterised in that a test strip which comprises nitro blue tetrazolium chloride as tetrazolium salt is used in step c).

3. Method according to claim 1, characterised in that the sample provided in step a) is a beverage.

4. Method according to claim 1, characterised in that the reagent composition from step e) has a pH between 2.5 and 5.5.

5. Method according to claim 1, characterised in that the determination of the acetate content in step g) is carried out reflectometrically.

6. Test kit for the determination of the acetate content of a sample, at least comprising
a) a buffer solution for dilution of the sample and setting of the pH, which comprises at least a buffer substance and malic acid.
b) a test strip which comprises at least a tetrazolium salt, ATP, coenzyme A, NAD, malate dehydrogenase, citrate synthase and acetyl CoA synthetase;
c) a liquid reagent composition which comprises at least phenazine methosulfate or a derivative thereof.

7. Test kit according to claim 6, characterised in that buffer solution, test strips and reagent compositions comprise, independently of one another, buffers and stabilisers.

8. Test kit according to claim 6, characterised in that the test strip comprises nitro blue tetrazolium chloride as tetrazolium salt.

9. Test kit according to claim 6, characterised in that the reagent composition has a pH between 2.5 and 5.5.
